SEQUENCE LISTING (50 sequences)

SEQ ID NO: 1 - protein, 44 aa, human
  VARIANT (1)...(1): Xaa = Tyr or His
XXDAIFTXSYRKXLXQLXARKLLXXIXXRQQGESNQERGARARL

SEQ ID NO: 2 - protein, 8 aa, human
  DISULFID (3)...(3): Disulfide linkage with amino acid 1 (Cys) of SEQ ID NO 3
AGCKNFFW

SEQ ID NO: 3 - protein, 6 aa, human
  DISULFID (1)...(1): Disulfide linkage with amino acid 3 (Cys) of SEQ ID NO 2
CSXFTK

SEQ ID NO: 4 - protein, 31 aa, human
HAEGTFTSDVSSYLEGQAAKEFIAWLVKGRG

SEQ ID NO: 5 - protein, 34 aa, human
  VARIANT (1)...(1): Xaa = Ser or Ala
XVSEXQXMHNLGXHXXXXERXXWLXXKLQDVHXX

SEQ ID NO: 6 - protein, 39 aa, human
  VARIANT (13)...(13): Xaa = Val or Met
SYSMEHFRWGKPXGXKRRPXKVYPNXXXXEXXEXXXXEX

SEQ ID NO: 7 - protein, 49 aa, human
  VARIANT (3)...(3): Xaa = Tyr or Asp or Asn
YLXXXLGAPXPYPDPLEPXREVCELNPXCDELADHIGFQXAYXRXYGXV

SEQ ID NO: 8 - protein, 32 aa, human
  VARIANT (2)...(2): Xaa = Gly or Ser or Ala
CXXLSTCXLGXXXXXXXXXXTXXXXXXGXXXP

SEQ ID NO: 9 - protein, 41 aa, human
  VARIANT (22)...(22): Xaa = Ala or Pro
SEEPPISLDLTFHLLREVLEMXXAEQLAQQAHSNRKLMEII

SEQ ID NO: 10 - protein, 17 aa, human
YGGFLRRIRPKLKWDNQ

SEQ ID NO: 11 - protein, 31 aa, human
  VARIANT (7)...(7): Xaa = Ser or Pro
YGGFMTXEXSQTPLXTLFKNAIXKNXXKKGX

SEQ ID NO: 12 - protein, 34 aa, human
  VARIANT (1)...(1): Xaa = para-Glu or para-Gln
XLGXQXXXXXXADXXKKXXPXXEXEEXAYGWMDF

SEQ ID NO: 13 - protein, 33 aa, human
  VARIANT (7)...(7): Xaa = Ser or Thr
HADGSFXXXXXXXLDXXAXXXFXXWXXXTXXXX

SEQ ID NO: 14 - protein, 10 aa, human
  VARIANT (1)...(1): Xaa = pGlu or 5-oxoPro or Gln
XHWSYGLRPG

SEQ ID NO: 15 - protein, 13 aa, human
  ACETYLATION (1)...(1)
  AMIDATION (13)...(13)
SYSMEHFRWGKPV

SEQ ID NO: 16 - protein, 28 aa, human
  VARIANT (12)...(12): Xaa = Met or Ile
SLRRSSCFGGRXDRIGAQSXLGCNSFRY

SEQ ID NO: 17 - protein, 10 aa, human
  AMIDATION (10)...(10)
GNLWATGHFM

SEQ ID NO: 18 - protein, 36 aa, human
  AMIDATION (35)...(35)
YPSKPDNPGEDAPAEDMARYYSALRHYINLITRQRY

SEQ ID NO: 19 - protein, 33 aa, human
  VARIANT (1)...(1): Glu is para-Glu
EPLPDCCRQKTCSCRLYELLHGAGNHAAGILTL

SEQ ID NO: 20 - protein, 36 aa, human
  AMIDATION (36)...(36)
YPIKPEAPGEDASPEELNRYYASLRHYLNLVTRQRY

SEQ ID NO: 21 - protein, 27 aa, human
  AMIDATION (27)...(27)
HSDGTFTSELSRLREGARLQRLLQGLV

SEQ ID NO: 22 - protein, 28 aa, human
  AMIDATION (28)...(28)
HSDAVFTDNYTRLRKQMAVKKYLNSILN

SEQ ID NO: 23 - protein, 23 aa, human
GIGKFLHSAGKFGKAFVGEIMKS

SEQ ID NO: 24 - protein, 23 aa, human
GIGKFLHSAKKFGKAFVGEIMNS

SEQ ID NO: 25 - protein, 19 aa, human
KWKVFKKIEKVGQATQIAK

SEQ ID NO: 26 - protein, 35 aa, human
KWKVFKKIEKMGRNIRNGIVKAGPAIAVLGEAKAL

SEQ ID NO: 27 - protein, 11 aa, human
  AMIDATION (11)...(11)
RPLPQEFFGLM

SEQ ID NO: 28 - protein, 5 aa, human
YPFPG

SEQ ID NO: 29 - protein, 4 aa, human
  AMIDATION (4)...(4)
YPFF

SEQ ID NO: 30 - protein, 5 aa, human
APGPR

SEQ ID NO: 31 - protein, 5 aa, human
VPDPR

SEQ ID NO: 32 - protein, 42 aa, human
YAEGTFISDYSIAMDKIHQQDFVNWLLAQKGKKNDWKHNITQ

SEQ ID NO: 33 - protein, 113 aa, human
LPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVME

SEQ ID NO: 34 - protein, 9 aa, human
SAKELRCQC

SEQ ID NO: 35 - protein, 14 aa, human
GPVSAVLTELRCTC

SEQ ID NO: 36 - protein, 12 aa, human
EAEEDGDLQCLC

SEQ ID NO: 37 - protein, 11 aa, human
VPLSRTVRCTC

SEQ ID NO: 38 - protein, 11 aa, human
TPVVRKGRCSC

SEQ ID NO: 39 - protein, 11 aa, human
KPVSLSYRCPC

SEQ ID NO: 40 - protein, 11 aa, human
APLATELRCQC

SEQ ID NO: 41 - protein, 11 aa, human
FPMFKKGRCLC

SEQ ID NO: 42 - protein, 10 aa, human
SPYSSDTTPC

SEQ ID NO: 43 - protein, 11 aa, human
APLAADTPTAC

SEQ ID NO: 44 - protein, 11 aa, human
APMGSDPPTAC

SEQ ID NO: 45 - protein, 11 aa, human
QPDAINAPVTC

SEQ ID NO: 46 - protein, 11 aa, human
QPSDVSIPITC

SEQ ID NO: 47 - protein, 10 aa, human
QPVGINSTTC

SEQ ID NO: 48 - protein, 11 aa, human
QPDALDVPSTC

SEQ ID NO: 49 - protein, 9 aa, human
GPASVPTTC

SEQ ID NO: 50 - protein, 12 aa, human
GPYGANMEDSVC

(19) United States
(12) Patent Application Publication
(10) Pub. No.: US 2003/0204063 A1
(43) Pub. Date: Oct. 30, 2003

(54) Modified biological peptides with increased potency

(76) Inventors: Denis Gravel, St-Lambert (CA); Abdelkrim Habi, Anjou (CA); Thierry Abribat, Montreal (CA)

Correspondence Address: David S Resnick, Nixon Peabody, 101 Federal Street, Boston, MA, 02110, US

(21) Appl. No.: 10/343,654

(22) PCT Filed: Aug. 2, 2001
(86) PCT No.: PCT/CA01/01119

(51) Int. Cl. C07K 14/61

(52) U.S. Cl. 530/399

(57) ABSTRACT

The present invention is concerned with modified biological peptides providing increased potency, prolonged activity and/or increased half-life thereof. The modification is made via coupling through an amide bond with at least one conformationally rigid substituent, either at the N-terminal of the peptide, the C-terminal of the peptide, on a free amino or carboxyl group along the peptide chain, or at a plurality of these sites. Those peptides exhibit clinical usefulness for example in treating states of insulin resistance associated with pathologies such as type II diabetes.

### FIELD OF THE INVENTION

The present invention is concerned with modified peptides providing increased biological potency, prolonged activity and/or increased half-life thereof. The modification is made via coupling through an amide bond with at least one conformationally rigid substituent either at the N-terminal of the peptide, the C-terminal of the peptide, or a free amino or carboxyl group along the peptide chain, or at a plurality of these sites.

### BACKGROUND OF THE INVENTION

Most peptides are rapidly degraded in a serum medium and as a result, their metabolites may sometimes end up with little or no residual biological activity. To increase the activity of a peptide, various techniques have been proposed. One of them is to anchor a hydrophobic chain at the N- or C-terminal of the peptidic sequence or at other residues along the peptidic chain. This technique nevertheless has limitations. For example, if the peptide comprises a long peptidic chain, the fact that a small hydrophobic group is anchored to the N- or C-terminal does not necessarily result in an increased activity of the peptide so-modified.

For example, it is known that substituting OH for a more hydrophobic group like —NEt₂ at the C-terminal of a peptide sequence can result in a significantly increased specific activity. However, these results are contradicted by several publications, such as Muranichi et al. in Pharm. Res., 1991, 8, 649-652, which stresses the inefficiency of a lauroyl group as a hydrophobic group at the N-terminal to increase activity. Accordingly, there does not seem to be any general rule or conclusion concerning biological potency, duration of activity and/or half life, that can be derived as a result of the addition of substituents on a peptide chain, whether at the N- or C-terminal, or on certain residues along the peptidic chain.

U.S. Pat. No. 6,020,311 discloses a hydrophobic growth hormone-releasing factor (GRF) analog wherein a rigidified hydrophobic moiety is coupled to the GRF peptide via an amide bond at the N-terminal of the peptide. Such analog is said to have an improved anabolic potency with reduced dosage, and a prolonged activity. According to the teaching of this patent, however, the rigidified hydrophobic moiety always comprises a carbonyl group at one extremity, which means that an amide coupling thereof to the GRF can only take place at an amino site to form the required amide bond. The patent does not mention, suggest or imply that similar results could be obtained if the amide coupling was made at the C-terminal by replacing the carbonyl group on the rigidified hydrophobic moiety with an amino group. The patent does not further mention, suggest or imply that the amide coupling could take place elsewhere on the peptide chain.

Biochemistry 2001, 40, pages 2860 to 2869 describes an hydrophobic glucagon-like peptide-1 (GLP-1) analog wherein hexanoic acid, a rigidified hydrophobic moiety is coupled to the GLP-1 peptide at the N-terminal of the peptide. The results show that this analog exhibits a decreased affinity for the GLP-1 receptor, but an in vivo bioactivity similar to or slightly better than that of the wild type GLP-1, hypothetically because of increased resistance to serum degradation. According to this study, the linkage of acyl chains to His¹, amino-acid substitutions of Ala², and the addition of amino-acid sequences at the N-terminal of the molecule would be better strategies to increase the in vivo biological activity than anchoring rigidified hydrophobic chains. However, most of these strategies involve a modification of the amino-acid composition of the natural molecule, which might have negative safety consequences for clinical applications, including the risks for immunogenicity and side effects.

There is therefore a great need to develop peptides modified in a manner such that their activity will be increased, thereby improving their potency, i.e., greater resistance to serum degradation and/or from hyperagonistic properties, and/or is increasing their half-life without changing the amino-acid sequence that would be clinically safe and acceptable.

### SUMMARY OF THE INVENTION

In accordance with the present invention, there is now provided a peptide of formula Xₙ—R₁ wherein:

R₁ is a peptide sequence which cannot be the GRF sequence when X represents a trans-3-hexanoyl group attached at N-terminal position of the peptide sequence;

each X can be identical or independent from the others and is selected from the following list constituted by conformationally rigid moieties bearing:

a) a carboxy or an amino group for coupling with the peptide sequence via an amide bond at the N-terminal of the peptide sequence, the C-terminal of the peptide sequence, at an available carboxy or amino site on the peptide sequence chain, and combinations thereof; and

b) a carboxy group for coupling with the peptide sequence via an ester bond at an available hydroxy site on the peptide sequence chain, and combinations thereof;

wherein,

n is any digit between 1 to 5;

X being defined as:

i) a straight, substituted C₁-C₁₀ alkyl;

ii) a branched, substituted C₁-C₁₀ alkyl;

iii) a straight or branched, unsubstituted or substituted C₁-C₁₀ alkene;

iv) a straight or branched, unsubstituted or substituted C₁-C₁₀ alkyne;

v) an unsubstituted or substituted, saturated or unsaturated C₃-C₁₀ cycloalkyl or heterocycloalkyl wherein the heteroatom is O, S or N;

vi) an unsubstituted or substituted C₅-C₁₄ aryl or heteroaryl wherein the heteroatom is O, S or N;

wherein the substituent in the definitions i) to vi) comprises one or more

a) straight or branched C₁-C₆ alkyl;

b) straight or branched C₁-C₆ alkene;

c) straight or branched C₁-C₆ alkyne;

d) C₃-C₁₀ cycloalkyl or heterocycloalkyl wherein at least 2 carbon atoms are optionally connected to the C₁-C₁₀ alkyl, C₁-C₁₀ alkene, C₁-C₁₀ alkyne, C₃-C₁₀ cycloalkyl or heterocycloalkyl, and C₅-C₁₄ aryl or heteroaryl; or

e) C₅-C₁₄ aryl or heteroaryl wherein at least 2 carbon atoms of the aryl or heteroaryl are optionally connected to the C₁-C₁₀ alkyl, C₁-C₁₀ alkene, C₁-C₁₀ alkyne, C₃-C₁₀ cycloalkyl or heterocycloalkyl, and C₅-C₁₄ aryl or heteroaryl;

and any isomers thereof, including cis and trans configurations, epimers, enantiomers, diastereoisomers, and racemic mixtures.

The term “aryl” includes phenyl, naphthyl and the like; the term “heterocycloalkyl” includes tetrahydrofuranyl, tetrahydrothiophanyl, tetrahydrothiopyranyl, tetrahydropyranyl and partially dehydrogenated derivatives thereof, azetidinyl, piperidinyl, pyrrolidinyl, and the like; the term “heteroaryl” comprises pyridinyl, indolyl, furanyl, imidazolyl, thiophanyl, pyrrolyl, quinolinyl, isoquinolinyl, pyrimidinyl, oxazolyl, thiazolyl, isothiazolyl, isooxazolyl, pyrazolyl, and the like.

The expression “conformationally rigid moiety” means an entity having limited conformational, i.e., rotational, mobility about its single bonds. Such mobility is limited, for example, by the presence of a double bond, a triple bond, or a saturated or unsaturated ring, which have little or no conformational mobility. As a result, the number of conformers or rotational isomers is reduced when compared, for example, with the corresponding straight, unsubstituted and saturated aliphatic chain. The conformationally rigid moiety may be hydrophobic, although this is not a prerequisite.

According to a preferred embodiment of the present invention the peptide sequence is selected from the group consisting of Growth hormone releasing factor (GRF), Somatostatin, Glucagon-like peptide 1 (7-37), amide human (GLP-1), hGLP-1 (7-36) NH₂ Parathyroid hormone fragments such as (PTH 1-34), Adrenocorticotropic hormone (ACTH), Osteocalcin, Calcitonin, Corticotropin releasing factor, Dynorphin A, β-Endorphin, Big Gastrin-1, GLP-2, Luteinizing hormone-releasing hormone, Melanocyte Stimulating Hormone (MSH), Atrial Natriuretic Peptide, Neuromedin B, Human Neuropeptide Y, Human Orexin A, Human Peptide YY, Human Secretin, Vasoactive Intestinal peptide (VIP), Antibiotic peptides (Magainin 1, Magainin 2, Cecropin A, and Cecropin B), Substance P (SP), Beta Casomorphin-5, Endomorphin-2, Procolipase, Enterostatin, gastric inhibitory peptide, Chromogranin A, Vasostatin I & II, Procalcitonin, ProNCT, ProCGRP, IL8 (monocyte-derived), GCP-2, PF4, IP-10, MIG, SDF-1α, GRO-α, I-TAC, RANTES, LD78, MIP-1α, MCP-1, MCP-2, MCP-3, MCP-4, Eotaxin, MDC, and functional derivatives or fragments thereof.

### DETAILED DESCRIPTION OF THE INVENTION

The amino acids are identified in the present application by the conventional three-letter abbreviations as indicated below, which are as generally accepted in the peptide art as recommended by the IUPAC-IUB commission in biochemical nomenclature:

| | | | |
|---|---|---|---|
| Alanine | Ala | Leucine | Leu |
| Arginine | Arg | Lysine | Lys |
| Asparagine | Asn | Methionine | Met |
| Aspartic acid | Asp | Phenylalanine | Phe |
| Cyesteine | Cys | Proline | Pro |
| Glutamic acid | Glu | Serine | Ser |
| Glutamine | Gln | Threonine | Thr |
| Glycine | Gly | Tryptophan | Trp |
| Histidine | His | Tyrosine | Tyr |
| Isoleucine | Ile | Value | Val |

All the peptide sequences set out herein are written according to the generally accepted convention whereby the N-terminal amino acid is on the left and the C-terminal amino acid is on the right.

The present invention relates to the use of at least one conformationally rigid moiety, to produce a new family of peptides with enhanced pharmacological properties.

The modified peptides of the present invention are prepared according to the following general method, well known in the art of solid phase synthesis.

Conformationally rigid moieties comprising a carboxy group are used for anchoring to amino groups such as those found on the lysine side chain as well as the N-terminus of peptides. Those comprising an amino group are used for anchoring to carboxyl groups such as those found on the aspartic or glutamic acid side chains or the C-terminus of peptides. For such cases, the anchoring reaction is preferably performed on a solid phase support (Merrifield R. B. 1963, J. Am. Chem. Soc., 1963, 85, 2149 and J. Am. Chem. Soc., 1964, 86, 304) using Benzotriazole-1-yl-oxy-tris (dimethylamino) phosphonium hexafluorophosphate described by Castro in the article (B. Castro et al., 1975, Tetrahedron letters, Vol. 14:1219).

With respect to the anchoring dynamic, the preferred working temperatures are between 20° C. and 60° C. The anchoring reaction time in the case of the more hydrophobic moieties, varies inversely with temperature, and varies between 0.1 and 24 hours.

Synthesis steps were carried out by solid-phase methodology on a manual peptide synthesizer using the Fmoc strategy. Fmoc amino acids were supplied by Chem Impex International Inc. Chicago and other commercial sources. Sequential Fmoc chemistry using BOP as coupling reagent was applied to the PL-Wang resin (Polymer Laboratories, catalog number: 1463-4799) for the production of the C-terminal carboxylic acid.

Fmoc deprotections were accomplished with piperidine 20% solution in DMF in three consecutive steps. Always under nitrogen scrubbing, a first solution of piperidine 20% was used for 1 min. to remove the major part of the Fmoc protecting groups. Then, the solution was drained, and another fresh piperidine 20% solution was introduced this time for 3 min., drained again and finally another solution of piperidine 20% for 10 min. The peptide-resin was then washed 4 times successively with 50 mL of DMF under nitrogen scrubbing. After completion of the synthesis, the resin was well washed with DMF and DCM prior to drying.

Final cleavage of side chain protecting groups and peptide-resin bonds were performed using the following mixture: TFA, ethanedithiol, triisopropylsilane, thioanisole, phenol, water (92:1.66:1.66:1.66:1:2). A final concentration of 20 mL of cleavage cocktail per gram of dried peptide-resin was used to cleave the peptide from the resin. The cleavage reaction was performed at room temperature for 2 hours. The free peptide, now in solution in the TFA cocktail, was then filtered on a coarse fritted disk funnel. The resin was then washed 3 times with pure TFA. The peptide/TFA mixture was evaporated under vacuum on a Rotary evaporator, precipitated and washed with ether prior to its dissolution in water and freeze drying to eliminate the remaining traces of solvent and scavengers.

Coupling of the First Fmoc-Amino Acid to the Wang Resin

We used 4-alkoxybenzyl alcohol polystyrene (Wang resin) and 2 eq of the desired Fmoc-amino acid in DMF and let both products mix together under nitrogen scrubbing for 15 min at room temperature. Then 3.3 eq of pyridine and 2 eq of 2,6-dichlorobenzoylchloride were added successively and the reaction was carried out under nitrogen scrubbing for 15-20 hours. (Seiber P., 1987, Tetrahedron Letters, Vol. 28, No. 49, pp 6147-6150). After this reaction, the reaction vessel was drained and the resin washed 4 times successively with DMF under nitrogen scrubbing. Any remaining hydroxyl groups of the resin were benzoylated with 3 eq of benzoylchloride and pyridine in DCE (dichloroethane) for 2 hours.

Coupling of Each Remaining Amino Acid on the Growing Peptide

For each of the following Fmoc-amino acid we dissolved 3 eq of the Fmoc-amino acid with 3 eq of BOP (Benzotriazole-1-yl-oxy-tris (dimethylamino) phosphonium hexafluorophosphate) (B. Castro et al., 1975, Tetrahedron letters, Vol. 14:1219) in DMF, added the resulting solution to the resin in the reaction vessel, started the nitrogen scrubbing and added 6 eq of DIPEA (diisopropylethylamine) to start the coupling reaction. The coupling mixture was scrubbed under nitrogen for 60 min. in the reaction vessel; then drained from the vessel, the resin was washed 3 times successively with DMF and a qualitative ninhydrin test was performed to verify completion of the reaction.

The coupling of the Fmoc-L-Lys(Aloc)-OH (PerSeptive Biosystems, catalog number: GEN911209), Fmoc-L-Glu(OAl)-OH (PerSeptive Biosystems, catalog number: GEN911207) and Fmoc-L-Asp(OAl)-OH (PerSeptive Biosystems, catalog number: GEN911205) were carried out in the same way as for the Fmoc-amino acids as described above.

Deprotection of Allylic Groups

The peptide-resin (X mmol) was then introduced in DCM under nitrogen scrubbing and after 10 min. the PdCl₂(PPh₃)₂ (X mmol×0.05/0.05 eq) (palladium(II) bis-triphenylphosphine) was added to the mixture (Bürger H., Kilion W., J. Organometallics, 1969, 18:299). Then the (CH₃CH₂CH₂)₃SnH (X mmol×6/6 eq) (tributyltinhydride) was diluted in DCM and added dropwise to the peptide-resin suspension with an addition funnel over a period of 30 minutes. The reaction was continued for another 10 minutes then the vessel was drained from the cleavage mixture and right after the peptide-resin was washed 4 times with DCM and 4 times with DMF (Dangles O., Guibe F., Balavoine G., Lavielle S., Marquet A., 1987, J. Org. Chem., 52:4984).

Coupling of the Conformationally Rigid Acids and Alkylamines

The coupling of the conformationally rigid acids and amines to the side chains of the peptide-resin was conducted under the same conditions as those of the Fmoc-amino acids except that for these side chain modifications we used 10 equivalents of the rigid moieties and coupling reagent instead of 3.

The invention is not limited to any particular peptide sequence. Preferred peptide sequences R¹ comprise those with therapeutic properties, as well as functional derivatives or fragments thereof. The therapeutic properties of such peptides which may be used in accordance with the present invention include, without limitation, treatment of bone diseases including osteoporosis, postmenopausal osteoporosis and bone deposits, cancer treatment, regulating blood glucose, type II diabetes, treatment to enhance mucosal regeneration in patients with intestinal diseases, treatment for diseases related to inflammatory responses, obesity treatment, treatment for autism and pervasive development disorders, hyperproliferative skin conditions, aging, altering the proliferation of peripheral blood mononuclear cells, regulation of myometrial contractility and of prostaglandin release, stimulation of ACTH release, inhibition of interleukin-8 production, stimulation of acid release, enhancement of mucosal regeneration in patients with intestinal diseases, treatment for hormone-dependent diseases and conditions including for hormone-dependent cancers, modulation of melanocyte information process, involved in pressure and volume homeostasis, regulation of exocrine and endocrine secretions, smooth muscle contraction, feeding, blood pressure, blood glucose, body temperature and cell growth, regulation of food intake and energy balance, inhibition of cancer cell growth, stimulation of pancreatic secretion, or stimulate cell growth.

Growth Hormone Releasing Factor (GRW):

Xaa₁-Xaa₂-Asp-Ala-Ile-Phe-Thr-Xaa₈-Ser-Tyr-Arg-Lys-Xaa₁₃-Leu-Xaa₁₅-Gln-Leu- Xaa₁₈-Ala-Arg-Lys-Leu-Leu-Xaa₂₄-Xaa₂₅-Ile-Xaa₂₇-Xaa₂₈-Arg-Gln-Gln-Gly-Glu-Ser- Asn-Gln-Glu-Arg-Gly-Ala-Arg-Ala-Arg-Leu-NH₂

wherein,

Xaa₁ is Tyr or His;

Xaa₂ is Val or Ala;

Xaa₈ is Asn or Ser;

Xaa₁₃ is Val or Ile;

Xaa₁₅ is Ala or Gly;

Xaa₁₈ is Ser or Tyr;

Xaa₂₄ is Gln or His;

Xaa₂₅ is Asp or Glu;

Xaa₂₇ is Met, Ile or Ile; and

Xaa₂₈ is Ser or Asn.

Somatostatin:

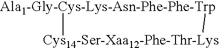

wherein,

Xaa₁₂ is Tyr or Ser.

Glucagon-Like Peptide 1 (7-37), (Amide Human (hGLP-1)):

His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-OH(NH₂)

Parathyroid Hormone Fragments (PTH 1-34):

Xaa₁-Val-Ser-Glu-Xaa₅-Gln-Xaa₇-Met-His-Asn-Leu-Gly-Xaa₁₃-His-Xaa₁₅-Xaa₁₆- Xaa₁₇-Xaa₁₈-Glu-Arg-Xaa₂₁-Xaa₂₂-Trp-Leu-Xaa₂₅-Xaa₂₆-Lys-Leu-Gln-Asp-Val-His- Xaa₃₃-Xaa₃₄-NH₂

wherein,

Xaa₁ is Ser or Ala;

Xaa₅ is Ile or Met;

Xaa₇ is Leu or Phe;

Xaa₁₃ is Lys or Glu;

Xaa₁₅ is Leu or Arg;

Xaa₁₆ is Asn or Ala or Ser or His;

Xaa₁₇ is Ser of Thr;

Xaa₁₈ is Met or Val or Leu;

Xaa₂₁ is Val or met or Gln;

Xaa₂₂ is Glu or Gln or Asp;

Xaa₂₅ is Arg or Gln;

Xaa₂₆ is Lys or Met;

Xaa₃₃ is Asn or Ser; and

Xaa₃₄ is Phe or Ala.

Adrenocorticotropic Hormone (ACTH):

Ser-Tyr-Ser-Met-Glu-His-Phe-Arg-Trp-Gly-Lys-Pro-Xaa₁₃-Gly-Xaa₁₅-Lys-Arg-Arg- Pro-Xaa₂₀-Lys-Val-Tyr-Pro-Asn-Xaa₂₆-Xaa₂₇-Xaa₂₈-Xaa₂₉-Glu-Xaa₃₁-Xaa₃₂-Glu- Xaa₃₄-Xaa₃₅-Xaa₃₆-Xaa₃₇-Glu-Xaa₃₉-NH₂

wherein,

Xaa₁₃ is Val or Met;

Xaa₁₅ is Lys or Arg;

Xaa₂₀ is Val or Ile;

Xaa₂₆ is Gly or Ser;

Xaa₂₇ is Ala or Phe or Val;

Xaa₂₈ is Glu or Gln;

Xaa₂₉ is Asp or Asn or Glu;

Xaa₃₁ is Ser or Thr;

Xaa₃₂ is Ala or Val or Ser;

Xaa₃₄ is Ala or Asn or Gly;

Xaa₃₅ is Phe or Met;

Xaa₃₆ is Pro or Gly;

Xaa₃₇ is Leu or Val or Pro; and

Xaa₃₉ is Phe or Val or Leu.

Osteocalcin:

Tyr-Leu-Xaa₅₂-Xaa₅₃-Xaa₅₄-Leu-Gly-Ala-Pro-Xaa₅₉-Pro-Tyr-Pro-Asp-Pro-Leu-Glu- Pro-Xaa₆₈-Arg-Glu-Val-Cys-Glu-Leu-Asn-Pro-Xaa₇₇-Cys-Asp-Glu-Leu-Ala-Asp- His-Ile-Gly-Phe-Gln-Xaa₈₉-Ala-Tyr-Xaa₉₂-Arg-Xaa₉₄-Tyr-Gly-Xaa₉₇-Val-NH₂

wherein,

Xaa₅₂ is Tyr or Asp or Asn;

Xaa₅₃ is Gln or His or Asn;

Xaa₅₄ is Trp or Gly;

Xaa₅₉ is Val or Ala;

Xaa₆₈ is Arg or Lys or His;

Xaa₇₇ is Asp or Asn;

Xaa₈₉ is Glu or Asp;

Xaa₉₂ is Arg or Lys;

Xaa₉₄ is Phe or Ile; and

Xaa₉₇ is Pro or Thr.

Calcitonin:

Cys-Xaa₈₆-Xaa₈₇-Leu-Ser-Thr-Cys-Xaa₉₂-Leu-Gly-Xaa₉₅-Xaa₉₆-Xaa₉₇-Xaa₉₈-Xaa₉₉- Xaa₁₀₀-Xaa₁₀₁-Xaa₁₀₂-Xaa₁₀₃-Xaa₁₀₄-Thr-Xaa₁₀₆-Xaa₁₀₇-Xaa₁₀₈-Xaa₁₀₉- Xaa₁₁₀-Xaa₁₁₁-Gly-Xaa₁₁₃-Xaa₁₁₄-Xaa₁₁₅-Pro-NH₂

wherein,

Xaa₈₆ is Gly or Ser or Ala;

Xaa₈₇ is Asn or Ser;

Xaa₉₂ is Met or Val;

Xaa₉₅ is Thr or Lys;

Xaa₉₆ is Tyr or Leu;

Xaa₉₇ is Thr or Ser;

Xaa₉₈ is Gln or Lys;

Xaa₉₉ is Asp or Glu;

Xaa₁₀₀ is Phe or Leu;

Xaa₁₀₁ is Asn or His;

Xaa₁₀₂ is Lys or Asn;

Xaa₁₀₃ is Phe or Leu;

Xaa₁₀₄ is His or Gln;

Xaa₁₀₆ is Phe or Tyr;

Xaa₁₀₇ is Pro or Ser;

Xaa₁₀₈ is Gln or Gly or Arg;

Xaa₁₀₉ is Thr or Ile;

Xaa₁₁₀ is Ala or Gly or Ser or Asp or Asn;

Xaa₁₁₁ is Ile or Phe or Val or Thr;

Xaa₁₁₃ is Val or Ala or Ser;

Xaa₁₁₄ is Gly or Glu; and

Xaa₁₁₅ is Ala or Thr.

Corticotropin Releasing Factor:

Ser-Glu-Glu-Pro-Pro-Ile-Ser-Leu-Asp-Leu-thr-Phe-His-Leu-Leu-Arg-Glu-Val-Leu- Glu-Met-Xaa₁₀₁-Xaa₁₀₂-Ala-Glu-Gln-Leu-Ala-Gln-Gln-Ala-His-Ser-Asn-Arg-Lys-Leu-Met-Glu-Ile-Ile-NH₂

wherein,

Xaa₁₀₁ is Ala or Pro; and

Xaa₁₀₂ is Arg or Gly.

Dynorphin A:

H-Tyr-Gly-Gly-Phe-Leu-Arg-Arg-Ile-Arg-Pro-Lys-Leu-Lys-Trp-Asp-Asn-Gln-OH

β-Endorphin:

H-Tyr-Gly-Gly-Phe-Met-Thr-Xaa₂₄₃-Glu-Xaa₂₄₅-Ser-Gln-Thr-Pro-Leu-Xaa₂₅₁-Thr- Leu-Phe-Lys-Asn-Ala-Ile-Xaa₂₅₉-Lys-Asn-Xaa₂₆₂-Xaa₂₆₃-Lys-Lys-Gly-Xaa₂₆₇-OH

wherein,

Xaa₂₄₃ is Ser or Pro;

Xaa₂₄₅ is Lys or Arg;

Xaa₂₅₁ is Val or Met;

Xaa₂₅₉ is Ile or Val;

Xaa₂₆₂ is Ala or Thr or Ser or Val;

Xaa₂₆₃ is Tyr or His; and

Xaa₂₆₇ is Glu or Leu or Gln or His.

Big Gastrin-1:

pXaa₅₉-Leu-Gly-Xaa₆₂-Gln-Xaa₆₄-Xaa₆₅-Xaa₆₆-Xaa₆₇-Xaa₆₈-Xaa₆₉-Ala-Asp-Xaa₇₂- Xaa₇₃-Lys-Lys-Xaa₇₆-Xaa₇₇-Pro-Xaa₇₉-Xaa₈₀-Glu-Xaa₈₂-Glu-Glu-Xaa₈₅-Ala-Tyr-Gly- Trp-Met-Asp-Phe-NH₂

wherein,

Xaa₅₉ is Glu or Gln;

Xaa₆₂ is Pro or Leu;

Xaa₆₄ is Gly or Asp;

Xaa₆₅ is Pro or Ser;

Xaa₆₆ is Pro or Gln;

Xaa₆₇ is His or Gln;

Xaa₆₈ is Leu or Met or Phe or Gln;

Xaa₆₉ is Val or Ile;

Xaa₇₂ is Pro or Leu;

Xaa₇₃ is Ser or Ala;

Xaa₇₆ is Gln or Glu;

Xaa₇₇ is Gly or Arg;

Xaa₇₉ is Trp or Pro or Arg;

Xaa₈₀ is Leu or Val or Met;

Xaa₈₂ is Glu or Lys; and

Xaa₈₅ is Glu or Ala.

GLP-2:

His-Ala-Asp-Gly-Ser-Phe-Xaa₁₅₂-Xaa₁₅₃-Xaa₁₅₄-Xaa₁₅₅-Xaa₁₅₆-Xaa₁₅₇-Xaa₁₅₈-Leu-Asp- Xaa₁₆₁₋Xaa₁₆₂-Ala-Xaa₁₆₄-Xaa₁₆₅-Xaa₁₆₆-Phe-Xaa₁₆₈-Xaa₁₆₉-Trp-Xaa₁₇₁-Xaa₁₇₂- Xaa₁₇₃-Thr-Xaa₁₇₅-Xaa₁₇₆-Xaa₁₇₇-Xaa₁₇₈;

wherein,

Xaa₁₅₂ is Ser or Thr;

Xaa₁₅₃ is Asp or Ser;

Xaa₁₅₄ is Glu or Asp;

Xaa₁₅₅ is Met or Phe;

Xaa₁₅₆ is Asn or Ser;

Xaa₁₅₇ is Thr or Lys;

Xaa₁₅₈ is Ile or Val or Ala;

Xaa₁₆₁ is Asn or Ile or His or Ser;

Xaa₁₆₂ is Leu or Lys;

Xaa₁₆₄ is Ala or Thr;

Xaa₁₆₅ is Arg or Gln or Lys;

Xaa₁₆₆ is Asp or Glu;

Xaa₁₆₈ is Ile or Leu;

Xaa₁₆₉ is Asn or Asp;

Xaa₁₇₁ is Leu or Ile;

Xaa₁₇₂ is Ile or Leu;

Xaa₁₇₃ is Gln or Asn or His;

Xaa₁₇₅ is Lys or Pro;

Xaa₁₇₆ is Ile or Val;

Xaa₁₇₇ is Thr or Lys; and

Xaa₁₇₈ is Asp or Glu.

Luteinizing Hormone-Releasing Hormone:

Xaa₁-His-Trp-Ser-Tyr-Gly-Leu-Arg-Pro-Gly-OH

wherein,

Xaa₁ is pGlu, 5-oxoPro or Gln.

Melanocyte Stimulating Hormone (MSH):

Ac-Ser-Tyr-Ser-Met-Glu-His-Phe-Arg-Trp-Gly-Lys-Pro-Val-NH₂

Atrial Natriuretic Peptide:

H-Ser-Leu-Arg-Arg-Ser-Ser-Cys-Phe-Gly-Gly-Arg-Xaa₁₃₅-Asp-Arg-Ile-Gly-Ala-Gln-Ser-Xaa₁₄₂-Leu-Gly-Cys-Asn-Ser-Phe-Arg-Tyr-OH

wherein,

Xaa₁₃₅ is Met or Ile; and

Xaa₁₄₂ is Gly or Ser.

Neuromedin B:

H-Gly-Asn-Leu-Trp-Ala-Thr-Gly-His-Phe-Met-NH₂

Human Neuropeptide Y:

H-Tyr-Pro-Ser-Lys-Pro-Asp-Asn-Pro-Gly-Glu-Asp-Ala-Pro-Ala-Glu-asp-Met-Ala- Arg-Tyr-Tyr-Ser-Ala-Leu-Arg-His-Tyr-Ile-Asn-Leu-Ile-Thr-Arg-Gln-Arg-Tyr-NH₂

Human Orexin A:

pGlu-Pro-Leu-Pro-Asp-Cys-Cys-Arg-Gln-Lys-Thr-Cys-Ser-Cys-Arg-Leu-Tyr-Glu- Leu-Leu-His-Gly-Ala-Gly-Asn-His-Ala-Ala-Gly-Ile-Leu-Thr-Leu-NH₂

Human Peptide YY:

H-Tyr-Pro-Ile-Lys-Pro-Glu-Ala-Pro-Gly-Glu-Asp-Ala-Ser-Pro-Glu-Glu-Leu-Asn- Arg-Tyr-Tyr-Ala-Ser-Leu-Arg-His-Tyr-Leu-Asn-Leu-Val-Thr-Arg-Gln-Arg-Tyr-NH₂

Human Secretin:

H-His-Ser-Asp-Gly-Thr-Phe-Thr-Ser-Glu-Leu-Ser-Arg-Leu-Arg-Glu-Gly-Ala-Arg-Leu-Gln-Arg-Leu-Leu-Gln-Gly-Leu-Val-NH₂

Vasoactive Intestinal Peptide (VIP):

H-His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn-NH₂

Antibiotic Peptides such as:

Magainin 1:
Gly-Ile-Gly-Lys-Phe-Leu-His-Ser-Ala-Gly-Lys-Phe-
Gly-Lys-Ala-Phe-Val-Gly-Glu-Ile-Met-Lys-Ser
Magainin 2:
Gly-Ile-Gly-Lys-Phe-Leu-His-Ser-Ala-Lys-Lys-Phe-
Gly-Lys-Ala-Phe-Val-Gly-Glu-Ile-Met-Asn-Ser
Cecropin A:
Lys-Trp-Lys-Val-Phe-Lys-Lys-Ile-Glu-Lys-Val-Gly-
Gln-Ala-Thr-Gln-Ile-Ala-Lys
Cecropin B:
Lys-Trp-Lys-Val-Phe-Lys-Lys-Ile-Glu-Lys-Met-Gly-
Arg-Asn-Ile-Arg-Asn-Gly-Ile-Val-Lys-Ala-Gly-Pro-
Ala-Ile-Ala-Val-Leu-Gly-Glu-Ala-Lys-Ala-Leu.
Substance P (SP):
Arg-Pro-Leu-Pro-Gln-Glu-Phe-Phe-Gly-Leu-Met-amide
Beta Casomorphin-5:
Tyr-Pro-Phe-Pro-Gly
Endomorphin-2:
Tyr-Pro-Phe-Phe-NH2
Procolipase:
100 aa peptide (X1-Pro-X2-Pro-Arg . . . )
Enterostatin
Val-Pro-Asp-Pro-Arg
Gastrin Inhibitory Peptide:
Tyr-Ala-Glu-Gly-Thr-Phe-Ile-Ser-Asp-Tyr-Ser-Ile-
Ala- Met-Asp-Lys-Ile-His-Gln-Gln-Asp-Phe- Val-
Asn-Trp-Leu- Leu-Ala-Gln-Lys-Gly-Lys-Lys-Asn-Asp-
Trp-Lys-His-Asn-Ile-Thr-Gln

Chromogranin A

Vasostatin I

Vasostatin II:

Leu Pro Val Asn Ser Pro Met Asn Lys Gly Asp Thr
Glu Val Met Lys Cys Ile Val Glu Val Ile Ser Asp
Thr Leu Ser Lys Pro Ser Pro Met Pro Val Ser Gln
Glu Cys Phe Glu Thr Leu Arg Gly Asp Glu Arg Ile
Leu Ser Ile Leu Arg His Gln Asn Leu Leu Lys Glu
Leu Gln Asp Leu Ala Leu Gln Gly Ala Lys Glu Arg
Ala His Gln Gln Lys Lys His Ser Gly Phe Glu Asp
Glu Leu Ser Glu Val Leu Glu Asn Gln Ser Ser Gln
Ala Glu Leu Lys Glu Ala Val Glu Glu Pro Ser Ser
Lys Asp Val Met Glu

Procalcitonin

ProNCT

ProCGRP

Chemokine Family:

CXC-Group:

IL8(Monocyte-Derived):

SerAlaLysGluLeuArgCysGlnCys . . .

GCP-2:

GlyProValSerAlaValLeuThrGluLeuArgCysThrCys . . .

PF4:

GluAlaGluGluAspGlyAspLeuGlnCysLeuCys . . .

IP-10:

ValProLeuSerArgThrValArgCCysThrCys . . .

MIG:

ThrProValValArgLysGlyArgCysSerCys . . .

SDF-1α:

LysProValSerLeuSerTyrArgCysProCys . . .

GROα:

AlaProLeuAlaThrGluLeuArgCysGlnCys . . .

I-TAC:

PheProMetPheLysLysGlyArgCysLeuCys . . .

CC-Group:

RANTES:

SerProTyrSerSerAspThrThrProCys . . .

LD78:

AlaProLeuAlaAlaAspThrProThrAlaCys . . .

MIP-1α:

AlaProMetGlySerAspProProThrAlaCys . . .

MCP-1:

GlnProAspAlaIleAsnAlaProValThrCys . . .

MCP-2:

GlnProSerAspValSerIleProIleThrCys . . .

MCP-3:

GlnProValGlyIleTAsnSeerThrThrCys . . .

MCP-4:

GlnProAspAlaLeuAspValProSerThrCys . . .

Eotaxin:

GlyProAlaSerValProThrThrCys . . .

MDC:

GlyProTyrGlyAlaAsnMetGluAspSerValCys . . .

and functional derivatives or fragments thereof.

The complete definition of the previously listed sequences are known inter alia from Mentlein, R (1999) Regul. Pept. 85:9-24 and from De Meester, I. Et al. (2000) Adv ExpMed Biol. 477:67-87. Those documents are incorporated by reference to the present application.

In a more preferred embodiment, the peptide is substituted with one or more conformationally rigid moieties. Preferred structures of the conformationally rigid moieties comprise those with a double bond, a triple bond or a saturated or unsaturated ring.

The following is a brief list of the formula of preferred conformationally rigid moieties, identified as Formula 1 to 63, which are suitable for the purposes of the present invention.

Among the preferred modified peptides according to the present invention, are those wherein the peptide sequence is the sequence of a natural peptide.

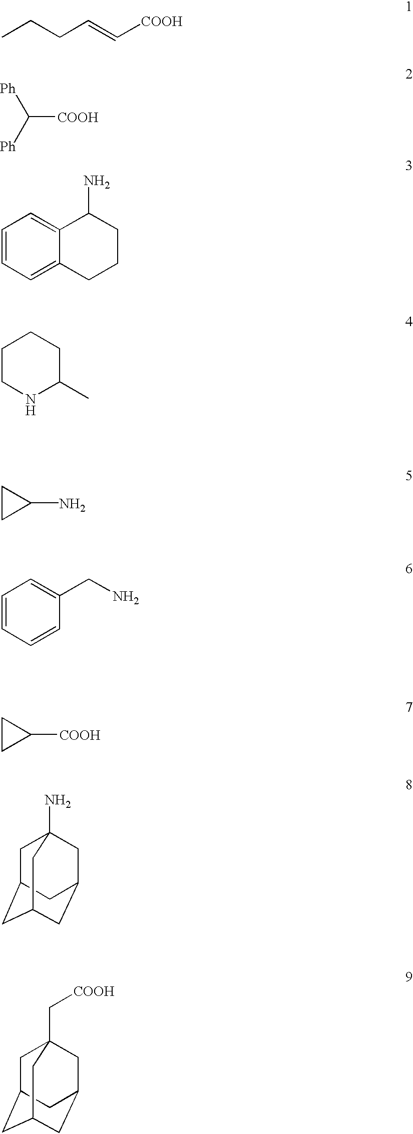

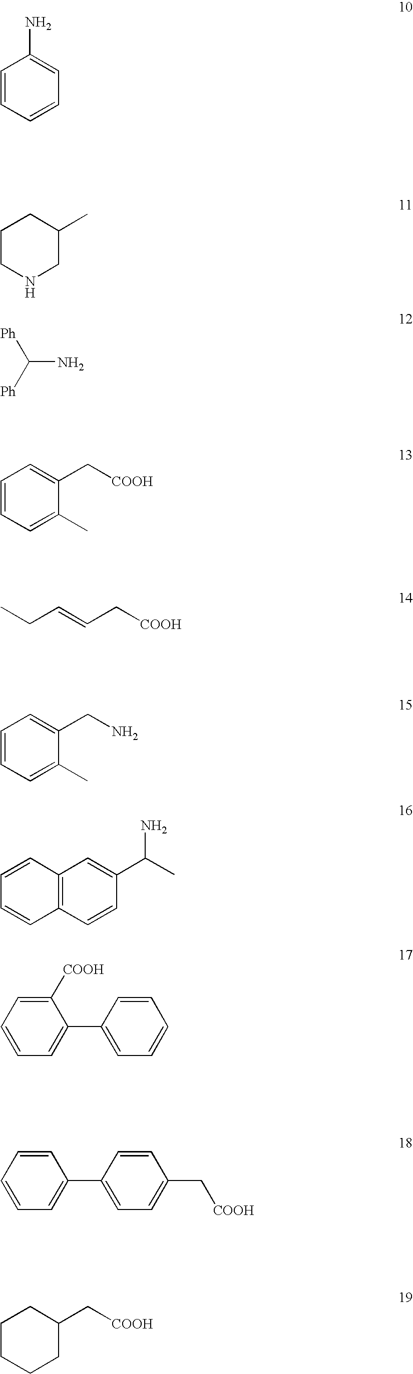

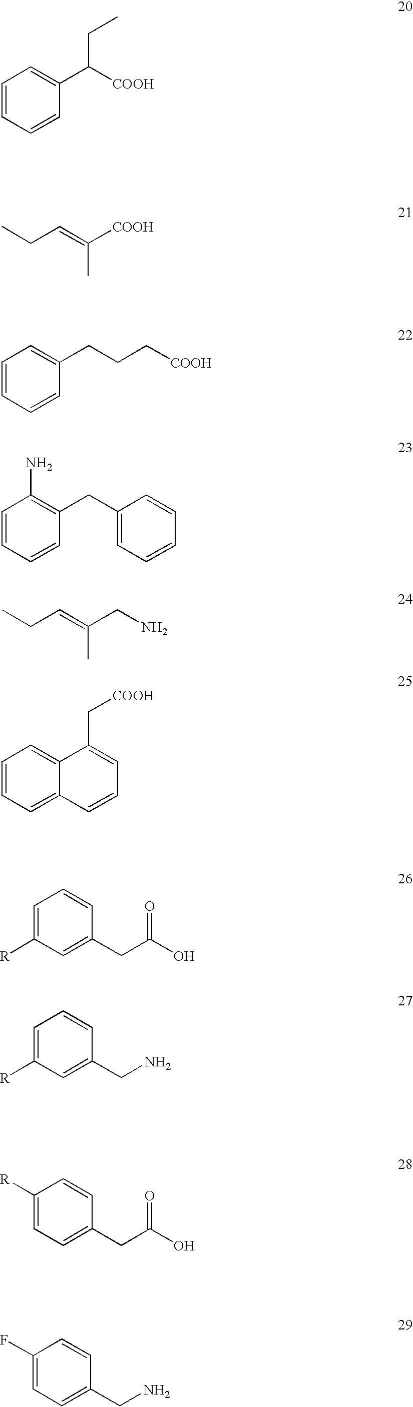

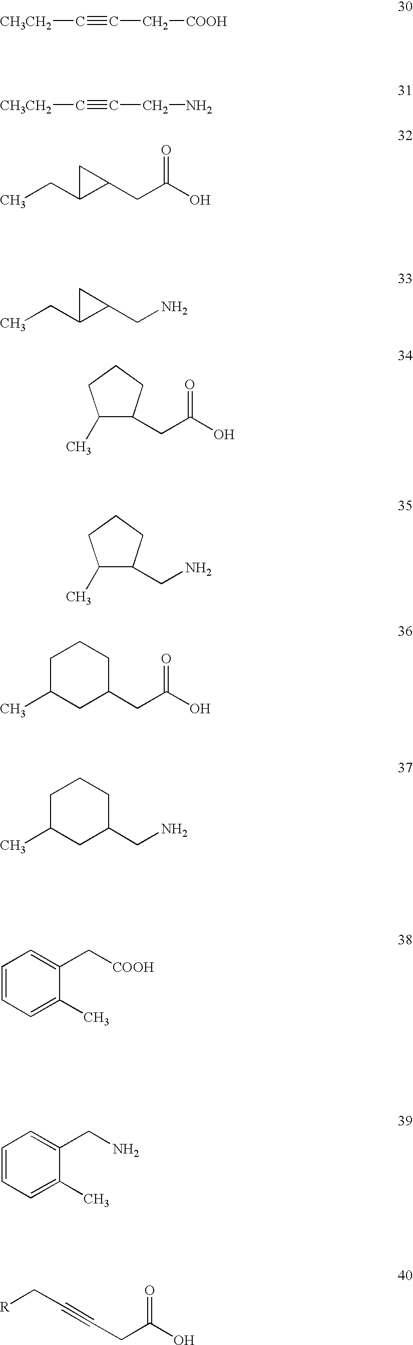

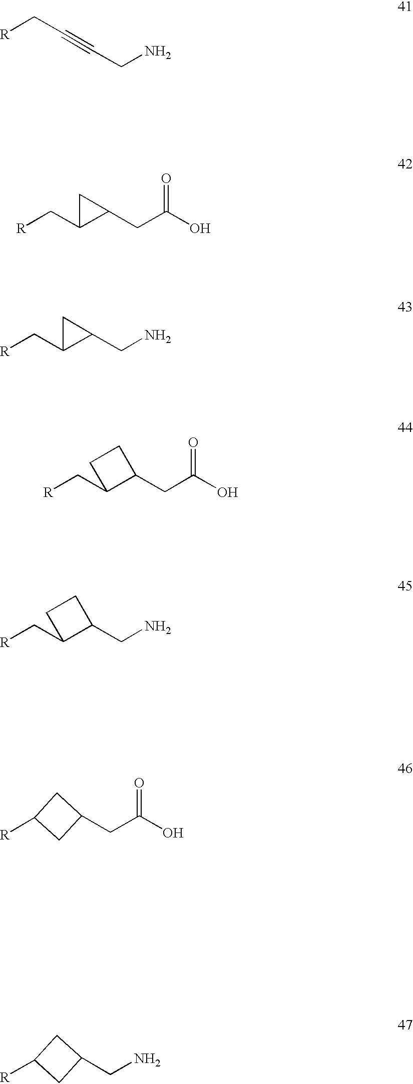

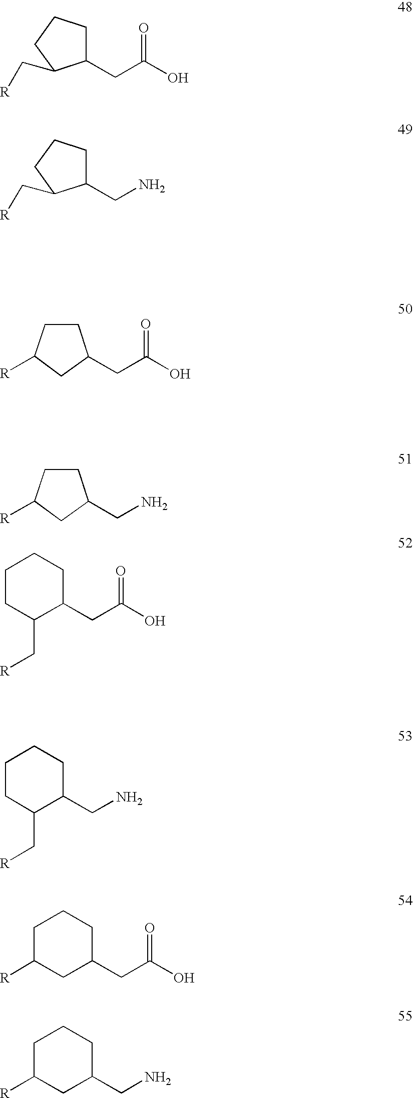

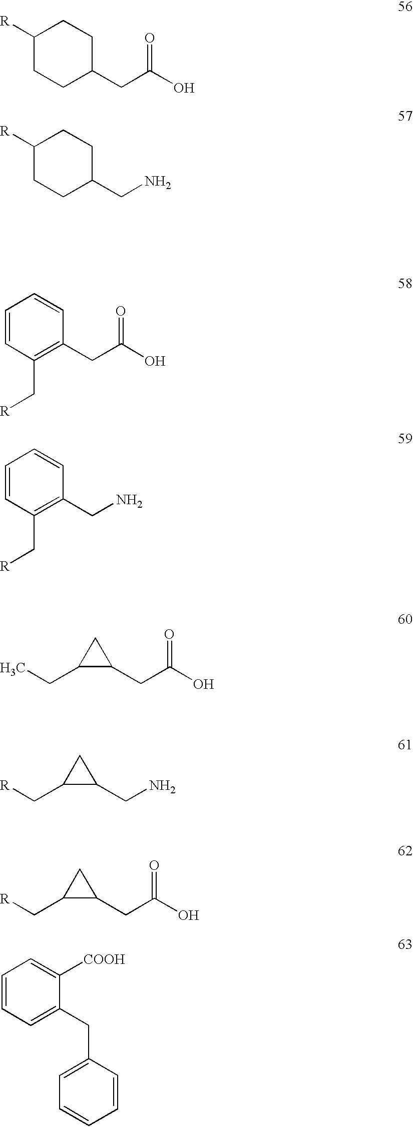

wherein, R is hydrogen, CH₃ or CH₂CH₃.

A preferred embodiment of the present invention is constituted by peptides wherein the peptide sequence is Somatostatin and at least one conformationally rigid moiety is coupled with said somatostatin peptide sequence via an amide bond at different positions as follows:

| Position | conformationally rigid moieties | |
|---|---|---|
| Ala₁ | 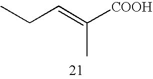 | |
| Asp₅ | 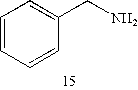 | 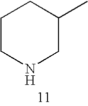 |
| Cys₁₄ | 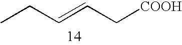 | |
| Ala₁ + Cys₁₄ | 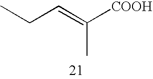 | 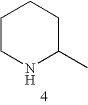 |

An another preferred embodiment of the present invention is constituted by those peptides wherein the peptide sequence is PTH 1-34 and at least one conformationally rigid moiety is coupled with said PTH 1-34 peptide sequence via an amide bond at different positions as follows:

| Position | conformationally rigid moieties | |
|---|---|---|
| Ser₁ | 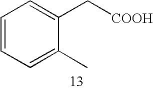 | 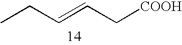 |
| Glu₄ | 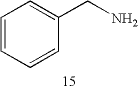 | 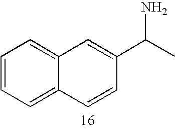 |
| Lys₂₆ | 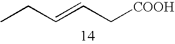 | 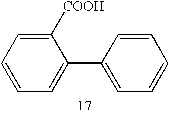 |
| Lys₂₇ | 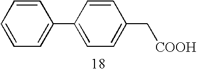 | 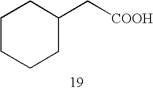 |
| Asp₃₀ | 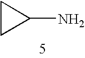 | 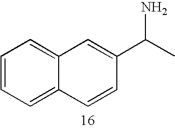 |
| Ser₁ +Lys₂₇ | 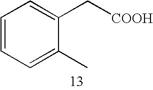 | 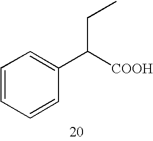 |

A further preferred embodiment of the present invention is constituted by those peptides wherein the peptide sequence is GLP-1 and at least one conformationally rigid moiety is coupled with said GLP-1 peptide sequence via an amide bond at different positions as follows:

| Position | conformationally rigid moieties | |
|---|---|---|
| His₁ | 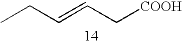 | 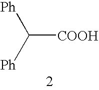 |
| | 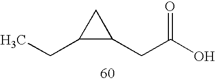 | 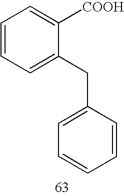 |
| | 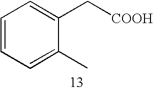 | |
| Glu₃ | 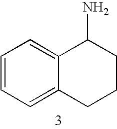 | 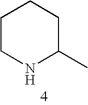 |
| Asp₉ | 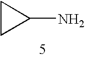 | 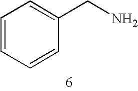 |
| His₁ + Glu₃ | 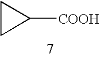 | 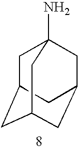 |
| His₁ + Asp₉ | 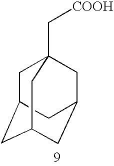 | 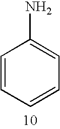 |
| Glu₃ + Asp₉ | 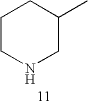 | 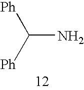 |

Also preferred among the modified peptides according to the invention are those peptides wherein;

the peptide sequence is GLP-2 and at least one conformationally rigid moiety is coupled with said GLP-2 peptide sequence via an amide or ester bond at different positions of the peptide sequence;

the peptide sequence is Enterostatin and at least one conformationally rigid moiety is coupled with said Enterostatin peptide sequence via an amide bond at different positions of the peptide sequence;

the peptide sequence is NPY and at least one conformationally rigid moiety is coupled with said NPY peptide sequence via an amide or ester bond at different positions of the peptide sequence;

the peptide sequence is NPYY and at least one conformationally rigid moiety is coupled with said NPYY peptide sequence via an amide or ester bond at different positions of the peptide sequence;

the peptide sequence is Secretin and at least one conformationally rigid moiety is coupled with said Secretin peptide sequence via an amide or ester bond at different positions of the peptide sequence;

the peptide sequence is Vasoactive Intestinal Peptide and at least one conformationally rigid moiety is coupled with said Vasoactive Intestinal Peptide sequence via an amide or ester bond at different positions of the peptide sequence;

the peptide sequence is Gastrin Inhibitory Peptide and at least one conformationally rigid moieties is coupled with said Gastrin inhibitory Peptide sequence via an amide or ester bond at different positions of the peptide sequence;

the peptide sequence is Vasostatin II and at least one conformationally rigid moiety is coupled with said Vasostatin II peptide sequence via an amide or ester bond at different positions of the peptide sequence;

the peptide sequence is RANTES and at least one conformationally rigid moiety is coupled with said RANTES peptide sequence via an amide or ester bond at different positions of the peptide sequence;

the peptide sequence is Eotaxin and at least one conformationally rigid moiety is coupled with said Eotaxin peptide sequence via an amide or ester bond at different positions of the peptide sequence.

In the modified peptides of the invention, the conformationally rigid moiety is preferably coupled with said peptide sequence via an amide bond at the N-terminal.

The modified peptides according to the invention, wherein the conformationally rigid moiety is the formula referenced 60 in the description, are of a particular interest.

The modified peptides of the present invention can be administered in various ways, such as for example, intravenously, subcutaneously, intradermally, transdermally, intraperitoneally, orally, or topically. The modified peptides of the present invention can also be administered by inhalation, when in a powder form or aerosol form. Furthermore, pharmaceutically acceptable carriers for delivery of modified peptides of the present invention include, without limitation, liposome, nanosome, patch, implant or any delivery devices.

In addition to the carboxy and amino groups present at the C- and N-terminals respectively of the peptide, other carboxy and amino sites can be available on the peptide chain. For example, if the peptide chain comprises amino acids provided with a carboxylic acid side chain such as aspartic acid and glutamic acid, additional carboxy sites will therefore be available on the chain for amidation. Should the peptide chain comprise amino acids with a carboxamide side chain such as asparagine and glutamine, these also provide additional carboxy groups for amidation by a conformationally rigid moiety, provided that they are accessed synthetically via the corresponding aspartic and glutamic acids. Further, if the peptide comprises amino acids provided with a basic side chain such as arginine, histidine or lysine, additional amino sites will then be available on the chain for amidation by a conformationally rigid moiety. The peptide chain may also include both acidic and basic amino acids, meaning that the conformationally rigid substituents could be coupled to the peptide chain via the N-terminal, the C-terminal, a carboxy site on the peptide chain, an amino site on the peptide chain, or a plurality of these sites.

The present invention will be more readily understood by referring to the following examples which are given to illustrate the invention rather than to limit its scope.

### EXAMPLE 1

Synthesis of GLP-1 Analogs

In accordance with the present invention, at least one of the following conformationally rigid moiety is coupled with the GLP-1 peptide sequence via an amide bond at different positions as follows.

| Position | conformationally rigid moieties | |
|---|---|---|
| His₁ | 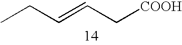 | 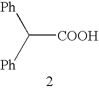 |
| | 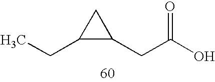 | 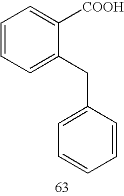 |
| | 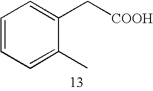 | |

hGLP-1 (7-37) Analogs Synthesis

hGLP-1 (7-37) derivatives modified at the amino terminus with rigid hydrophobic moieties were synthesized using Fmoc chemistry (1), on the Symphony apparatus (Rainin Instrument Co., Inc.). Fmoc-Gly-Wang resin (0.70 mmole/g) and five equivalents of reagents (100 μm scale, amino acids concentration of 200 mM), were used with a time coupling of 30 minutes. The reactions have been monitored by the Kaiser test. The three conformationally rigid moieties introduced at the N-terminus of the hGLP-1 (7-37) are:

Peptide # 1=(O-Tolylacetic acid-His⁷)-hGLP-1 (7-37) [O-Tolylacetic acid (13) (10 equivalents per coupling; coupling time 45 min)]

Peptide # 2=((+,−)-cis-2-Ethylcyclopropylacetic acid -His⁷)-hGLP-1 (7-37) [(+,−)-cis-2-Ethylcyclopropylacetic acid (60) (7.5 equivalents per coupling: coupling time 60 min)].

The peptides were cleaved using a TFA cocktail (92% TFA, 2% ethanedithiol, 2% thioanisole, 2% triisopropylsilane, 2% water, 2% (w/v) phenol) for 2 hours. All the analogs have been purified by reverse-phase HPLC. They have been analyzed by analytical HPLC and by MS (MALDI-TOF).

The synthesis of GLP-1 analogs is well known to the person skilled in the art and is fuirther illustrated by the general references Fmoc Solid Phase Peptide Synthesis. A Practical Approach (2000). Chan, W. C. and White, P. D., Oxford University Press, New York, USA, 346p which are incorporated by reference.

Biological Assess of GLP-1 Analogs

Materials & Methods

Oral Glucose Tolerance Test (OGTT)

Six-week old female CD1 mice (Charles River) were fasted for at least 16 hours. Mice were given 1.5 mg of glucose per gram of body weight orally in water through a gastric savage tube at t=o min and blood was collected from a tail vein at t=0, 10, 20, 30, 60, 90 and 120 min for measurement of blood glucose using a glucose meter (Lifescan). Peptides or vehicle were injected subcutaneously 5 min prior to the glucose administration. Data were expressed as the area under the curve, calculated from the change (delta) in blood glucose for each time, using the trapezoidal rule. Therefore, the data represent the integrated increase in blood glucose over a 120 min period following glucose administration. Data presented are the mean±SEM of 4 to 11 animals per group.

Test Articles

All peptides, including wild-type GLP-1 (7-37), were tested in the OGTT test at 3 different concentrations: 1, 5 and 10 ug per mouse. In a first set of experiments (study A), peptide 3 was tested in comparison with vehicle and hGLP-1(7-37). In a second set of experiments (study B), peptides 1 and 2 were tested in comparison with vehicle and hGLP-1 (7-37).

wt GLP1: hGLP(7-37)

Peptide #1: (O-Tolylacetic acid-His⁷)-hGLP-1 (7-37)

Peptide #2: ((+,−)-cis-2-Ethylcyclopropylacetic acid-His⁷)-hGLP-1 (7-37)

Peptide #3: (Hexanoyl-trans-3-His⁷)-hGLP-1 (7-37)

Results and Conclusions

Results are shown in FIG. I (study A) and FIG. II (study B)

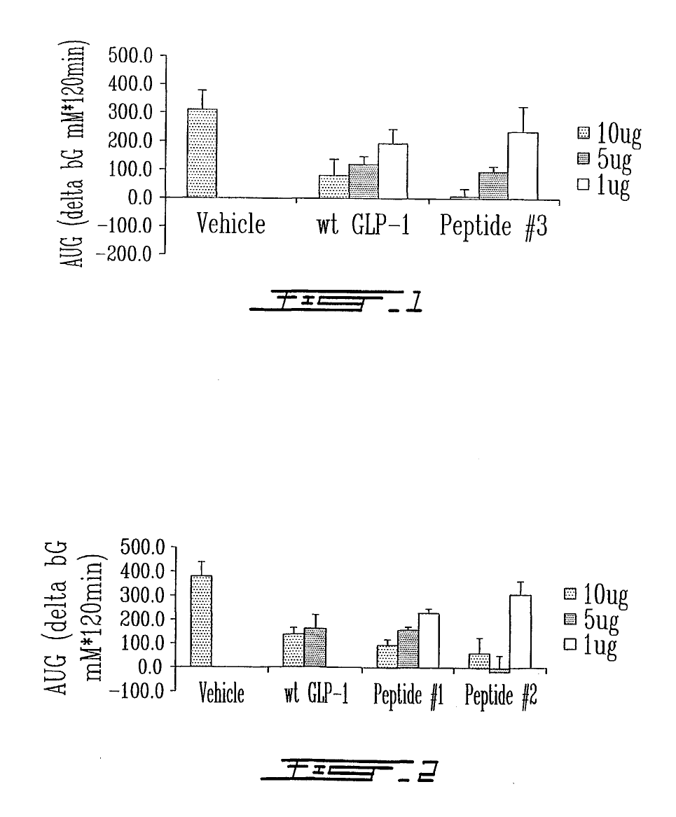

In studies A and B, administration of vehicle resulted in a similar integrated response in glucose levels (study A: 380±57 vs study B: 309±68 mM×120 min), illustrating the validity and reproducibility of the methodology. Although wt GLP-1 induced a dose-related decrease in the glucose response, this peptide was not able to completely suppress the glucose response at any dose, which might be interpreted as a limitation in its potential clinical usefulness. In contrast, peptide 3 (study A, FIG. 1) was able to completely abolish the glucose response, but only at the 10 ug dose (9±26 mM×120 min). Surprisingly, peptide 2 (study B, FIG. 2) was even more potent than peptide 3, being able to totally prevent the glucose response both at the 5 ug and the 10 ug doses (5 ug: −17±67 mM×120 min; 10 ug: 61±64 mM×120 min). In conclusion, the GLP-1 analog corresponding to peptide 2 was identified with marked increased biological potency over the wild type GLP-1 (7-37), because of this increased potency, this peptide may have clinical usefulness in treating states of insulin resistance associated with pathologies such as type II diabetes.

| Position | conformationally rigid moieties | |
|---|---|---|
| Glu₃ | 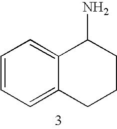 | 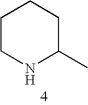 |
| Asp₉ | 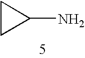 | 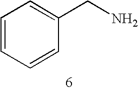 |
| His₁ + Glu₃ | 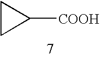 | 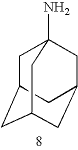 |
| His₁ + Asp₉ | 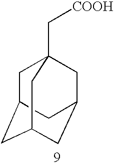 | 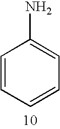 |
| Glu₃ + Asp₉ | 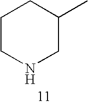 | 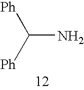 |

### EXAMPLE 2

PTH 1-34 Analogs

In accordance with the present invention, at least one of the following conformationally rigid moiety is coupled with the PTH 1-34 peptide sequence via an amide bond at different positions as follows.

| Position | conformationally rigid moieties | |
|---|---|---|
| Ser₁ | 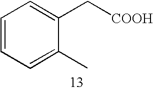 | 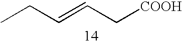 |
| Glu₄ | 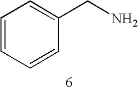 | 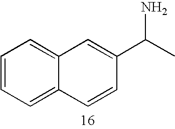 |
| Lys₂₆ | 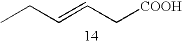 | 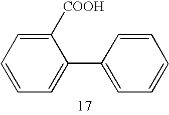 |
| Lys₂₇ | 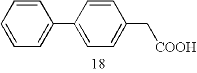 | 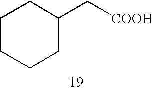 |
| Asp₃₀ | 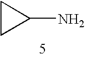 | 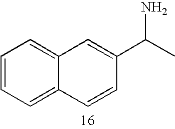 |
| Ser₁ +Lys₂₇ | 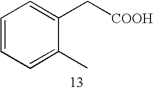 | 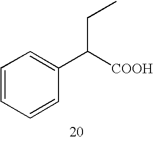 |

### EXAMPLE 3

Somatostatin Analogs

In accordance with the present invention, at least one of the following conformationally rigid moiety is coupled with the somatostatin peptide sequence via an amide bonds at different position as follows.

| Position | conformationally rigid moieties | |
|---|---|---|
| Ala₁ | 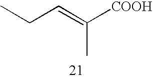 | |
| Asp₅ | 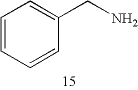 | 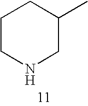 |
| Cys₁₄ | 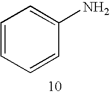 | |
| Ala₁ + Cys₁₄ | 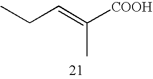 | 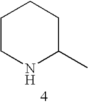 |

While the invention has been described in connection with specific embodiments thereof, it will be understood that it is capable of further modifications, and this application is intended to cover any variations, uses or adaptations of the invention following, in general, the principles of the invention, and including such departures from the present description as come within known or customary practice within the art to which the invention pertains, and as may be applied to the essential features hereinbefore set forth, and as follows in the scope of the appended claims.

What is claimed is:

1. A peptide of formula Xₙ—R₁ wherein:
R₁ is a peptide sequence, a functional analog thereof or a fragment thereof;
each X can be identical or independent from the others and is selected from the following list constituted by conformationally rigid moieties:
i) a straight, substituted C₁-C₁₀ alkyl;
ii) a branched, substituted C₁-C₁₀ alkyl;
iii) a straight or branched, unsubstituted or substituted C₁-C₁₀ alkene;
iv) a straight or branched, unsubstituted or substituted C₁-C₁₀ alkyne;
v) an unsubstituted or substituted, saturated or unsaturated C₃-C₁₀ cycloalkyl or heterocycloalkyl wherein the heteroatom is O, S or N;
vi) an unsubstituted or substituted C₅-C₁₄ aryl or heteroaryl wherein the heteroatom is O, S or N;
wherein the substituent in the definitions i) to vi) comprises one or more
a) straight or branched C₁-C₆ alkyl;
b) straight or branched C₁-C₆ alkene;
c) straight or branched C₁-C₆ alkyne;
d) C₃-C₁₀ cycloalkyl or heterocycloalkyl wherein at least 2 carbon atoms are optionally connected to the C₁-C₁₀ alkyl, C₁-C₁₀ alkene, C₁-C₁₀ alkyne, C₃-C₁₀ cycloalkyl or heterocycloalkyl, and C₅-C₁₄ aryl or heteroaryl; or
e) C₅-C₁₄ aryl or heteroaryl wherein at least 2 carbon atoms of the aryl or heteroaryl are optionally connected to the C₁-C₁₀ alkyl, C₁-C₁₀ alkene, C₁-C₁₀ alkyne, C₃-C₁₀ cycloalkyl or heterocycloalkyl, and C₅-C₁₄ aryl or heteroaryl, said group X also comprising at least one group selected from:
α) a carboxy or an amino group for coupling with the peptide sequence via an amide bond at the N-terminal of the peptide sequence, the C-terminal of the peptide sequence, at an available carboxy or amino site on the peptide sequence chain, and combinations thereof; and
β) a carboxy group for coupling with the peptide sequence via an ester bond at an available hydroxy site on the peptide sequence chain, and combinations thereof;
wherein,
n is any digit between 1 to 5;
and any isomers thereof, including cis and trans configurations, epimers, enantiomers, diastereoisomers, and racemic mixtures,
the peptides defined in claim 1 of U.S. Pat. No. 6,020,311 being excluded.

2. A peptide as claimed in claim 1 wherein the peptide sequence is selected from the group consisting of Growth hormone releasing factor (GRF), Somatostatin, Glucagon-like peptide 1 (7-37), amide human (GLP-1) hGLP-1 (7-36) NH₂, Parathyroid hormone fragments (PTH 1-34), Adrenocorticotropic hormone (ACTH), Osteocalcin, Calcitonin, Corticotropin releasing factor, Dynorphin A, β-Endorphin, Big Gastrin-1, GLP-2, Luteinizing hormone-releasing hormone, Melanocyte Stimulating Hormone (MSH), Atrial Natriuretic Peptide, Neuromedin B, Human Neuropeptide Y, Human Orexin A, Human Peptide YY, Human Secretin, Vasoactive Intestinal peptide (VIP), Antibiotic peptides (Magainin 1, Magainin 2, Cecropin A, and Cecropin B), Substance P (SP), Beta Casomorphin-5, Endomorphin-2, Procolipase, Enterostatin, gastric inhibitory peptide, Chromogranin A, Vasostatin I & II, Procalcitonin, ProNCT, CGRP (Calcitonin Gene Related Peptide), IL8 (monocyte-derived), GCP-2, PF4, IP-10, MIG, SDF-1α, GRO-α, I-TAC, RANTES, LD78, MIP-1α, MCP-1, MCP-2, MCP-3, MCP-4, Eotaxin, MDC, and functional analogs and derivatives or fragments thereof.

3. A peptide as claimed in claim 1 or 2 wherein the conformationally rigid moiety comprises at least a double bond, a triple bond or a saturated or unsaturated ring.

4. A peptide as claimed in any one of claims 1 to 3 wherein the conformationally rigid moiety comprises one or more of the structures of Formula 1 to 63 as defined in the description.

5. A peptide as claimed in any one of claims 1 to 4 wherein the peptide sequence is selected from the group consisting of:
Growth Hormone Releasing Factor (GRF):
Xaa₁-Xaa₂-Asp-Ala-Ile-Phe-Thr-Xaa₈-Ser-Tyr-Arg-Lys-Xaa₁₃-Leu-Xaa₁₅-Gln-Leu- Xaa₁₈-Ala-Arg-Lys-Leu-Leu-Xaa₂₄-Xaa₂₅-Ile-Xaa₂₇-Xaa₂₈-Arg-Gln-Gln-Gly-Glu-Ser- Asn-Gln-Glu-Arg-Gly-Ala-Arg-Ala-Arg-Leu-NH₂
wherein,
Xaa₁ is Tyr or His;
Xaa₂ is Val or Ala;
Xaa₈ is Asn or Ser;
Xaa₁₃ is Val or Ile;
Xaa₁₅ is Ala or Gly;
Xaa₁₈ is Ser or Tyr;
Xaa₂₄ is Gln or His;
Xaa₂₅ is Asp or Glu;
Xaa₂₇ is Met, Ile or Nle; and
Xaa₂₈ is Ser or Asn;
Somatostatin: 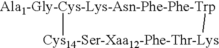
wherein,
Xaa₁₂ is Tyr or Ser;
Glucagon-Like Peptide 1 (7-37), (Amide Human (hGLP-1)):
His-Ala-Glu-Gly-Thr-Phe-Thr-Ser-Asp-Val-Ser-Ser-Tyr-Leu-Glu-Gly-Gln-Ala-Ala- Lys-Glu-Phe-Ile-Ala-Trp-Leu-Val-Lys-Gly-Arg-Gly-OH(NH₂)
Parathyroid Hormone Fragments (PTH 1-34):
Xaa₁-Val-Ser-Glu-Xaa₅-Gln-Xaa₇-Met-His-Asn-Leu-Gly-Xaa₁₃-His-Xaa₁₅-Xaa₁₆- Xaa₁₇-Xaa₁₈-Glu-Arg-Xaa₂₁-Xaa₂₂-Trp-Leu-Xaa₂₅-Xaa₂₆-Lys-Leu-Gln-Asp-Val-His- Xaa₃₃-Xaa₃₄-NH₂
wherein,
Xaa₁ is Ser or Ala;
Xaa₅ is Ile or Met;
Xaa₈ is Leu or Phe;
Xaa₁₃ is Lys or Glu;
Xaa₁₅ is Leu or Arg;
Xaa₁₆ is Asn or Ala or Ser or His;
Xaa₁₇ is Ser of Thr;
Xaa₁₈ is Met or Val or Leu;
Xaa₂₁ is Val or met or Gin;
Xaa₂₂ is Glu or Gln or Asp;
Xaa₂₅ is Arg or Gin;
Xaa₂₆ is Lys or Met;
Xaa₃₃ is Asn or Ser; and
Xaa₃₄ is Phe or Ala;
Adrenocorticotropic Hormone (ACTH):
Ser-Tyr-Ser-Met-Glu-His-Phe-Arg-Trp-Gly-Lys-Pro-Xaa₁₃-Gly-Xaa₁₅-Lys-Arg-Arg- Pro-Xaa₂₀-Lys-Val-Tyr-Pro-Asn-Xaa₂₆-Xaa₂₇-Xaa₂₈-Xaa₂₉-Glu-Xaa₃₁-Xaa₃₂-Glu- Xaa₃₄-Xaa₃₅-Xaa₃₆-Xaa₃₇-Glu-Xaa₃₉-NH₂
wherein,
Xaa₁₃ is Val or Met;
Xaa₁₅ is Lys or Arg;
Xaa₂₀ is Val or Ile;
Xaa₂₆ is Gly or Ser;
Xaa₂₇ is Ala or Phe or Val;
Xaa₂₈ is Glu or Gln;
Xaa₂₉ is Asp or Asn or Glu;
Xaa₃₁ is Ser or Thr;
Xaa₃₂ is Ala or Val or Ser;
Xaa₃₄ is Ala or Asn or Gly;
Xaa₃₅ is Phe or Met;
Xaa₃₆ is Pro or Gly;
Xaa₃₇ is Leu or Val or Pro; and
Xaa₃₉ is Phe or Val or Leu;
Osteocalcin:
Tyr-Leu-Xaa₅₂-Xaa₅₃-Xaa₅₄-Leu-Gly-Ala-Pro-Xaa₅₉-Pro-Tyr-Pro-Asp-Pro-Leu-Glu- Pro-Xaa₆₈-Arg-Glu-Val-Cys-Glu-Leu-Asn-Pro-Xaa₇₇-Cys-Asp-Glu-Leu-Ala-Asp- His-Ile-Gly-Phe-Gln-Xaa₈₉-Ala-Tyr-Xaa₉₂-Arg-Xaa₉₄-Tyr-Gly-Xaa₉₇-Val-NH₂
wherein,
Xaa₅₂ is Tyr or Asp or Asn;
Xaa₅₃ is Gln or His or Asn;
Xaa₅₄ is Trp or Gly;
Xaa₅₉ is Val or Ala;
Xaa₆₈ is Arg or Lys or His;
Xaa₇₇ is Asp or Asn;
Xaa₈₉ is Glu or Asp;
Xaa₉₂ is Arg or Lys;
Xaa₉₄ is Phe or Ile; and
Xaa₉₇ is Pro or Thr;
Calcitonin:
Cys-Xaa₈₆-Xaa₈₇-Leu-Ser-Thr-Cys-Xaa₉₂-Leu-Gly-Xaa₉₅-Xaa₉₆-Xaa₉₇-Xaa₉₈-Xaa₉₉- Xaa₁₀₀-Xaa₁₀₁-Xaa₁₀₂-Xaa₁₀₃-Xaa₁₀₄-Thr-Xaa₁₀₆-Xaa₁₀₇-Xaa₁₀₈-Xaa₁₀₉- Xaa₁₁₀-Xaa₁₁₁-Gly-Xaa₁₁₃-Xaa₁₁₄-Xaa₁₁₅-Pro-NH₂
wherein,
Xaa₈₆ is Gly or Ser or Ala;
Xaa₈₇ is Asn or Ser;
Xaa₉₂ is Met or Val;
Xaa₉₅ is Thr or Lys;
Xaa₉₆ is Tyr or Leu;
Xaa₉₇ is Thr or Ser;
Xaa₉₈ is Gln or Lys;
Xaa₉₉ is Asp or Glu;
Xaa₁₀₀ is Phe or Leu;
Xaa₁₀₁ is Asn or His;
Xaa₁₀₂ is Lys or Asn;
Xaa₁₀₃ is Phe or Leu;
Xaa₁₀₄ is His or Gln;
Xaa₁₀₆ is Phe or Tyr;
Xaa₁₀₇ is Pro or Ser;
Xaa₁₀₈ is Gln or Gly or Arg;
Xaa₁₀₉ is Thr or Ile;
Xaa₁₁₀ is Ala or Gly or Ser or Asp or Asn;
Xaa₁₁₁ is Ile or Phe or Val or Thr;
Xaa₁₁₃ is Val or Ala or Ser;
Xaa₁₁₄ is Gly or Glu; and
Xaa₁₁₅ is Ala or Thr;
Corticotropin Releasing Factor:
Ser-Glu-Glu-Pro-Pro-Ile-Ser-Leu-Asp-Leu-thr-Phe-His-Leu-Leu-Arg-Glu-Val-Leu- Glu-Met-Xaa₁₀₁-Xaa₁₀₂-Ala-Glu-Gln-Leu-Ala-Gln-Gln-Ala-His-Ser-Asn-Arg-Lys-Leu-Met-Glu-Ile-Ile-NH₂
wherein,
Xaa₁₀₁ is Ala or Pro; and
Xaa₁₀₂ is Arg or Gly;
Dynorphin A:
H-Tyr-Gly-Gly-Phe-Leu-Arg-Arg-Ile-Arg-Pro-Lys-Leu-Lys-Trp-Asp-Asn-Gln-OH
β-Endorphin:
H-Tyr-Gly-Gly-Phe-Met-Thr-Xaa₂₄₃-Glu-Xaa₂₄₅-Ser-Gln-Thr-Pro-Leu-Xaa₂₅₁-Thr- Leu-Phe-Lys-Asn-Ala-Ile-Xaa₂₅₉-Lys-Asn-Xaa₂₆₂-Xaa₂₆₃-Lys-Lys-Gly-Xaa₂₆₇-OH
wherein,
Xaa₂₄₃ is Ser or Pro;
Xaa₂₄₅ is Lys or Arg;
Xaa₂₅₁ is Val or Met;
Xaa₂₅₉ is Ile or Val;
Xaa₂₆₂ is Ala or Thr or Ser or Val;
Xaa₂₆₃ is Tyr or His; and
Xaa₂₆₇ is Glu or Leu or Gln or His;
Big Gastrin-1:
pXaa₅₉-Leu-Gly-Xaa₆₂-Gln-Xaa₆₄-Xaa₆₅-Xaa₆₆-Xaa₆₇-Xaa₆₈-Xaa₆₉-Ala-Asp-Xaa₇₂- Xaa₇₃-Lys-Lys-Xaa₇₆-Xaa₇₇-Pro-Xaa₇₉-Xaa₈₀-Glu-Xaa₈₂-Glu-Glu-Xaa₈₅-Ala-Tyr-Gly- Trp-Met-Asp-Phe-NH₂
wherein,
Xaa₅₉ is Glu or Gln;
Xaa₆₂ is Pro or Leu;
Xaa₆₄ is Gly or Asp;
Xaa₆₅ is Pro or Ser;
Xaa₆₆ is Pro or Gln;
Xaa₆₇ is His or Gln;
Xaa₆₈ is Leu or Met or Phe or Gln;
Xaa₆₉ is Val or Ile;
Xaa₇₂ is Pro or Leu;
Xaa₇₃ is Ser or Ala;
Xaa₇₆ is Gln or Glu;
Xaa₇₇ is Gly or Arg;
Xaa₇₉ is Trp or Pro or Arg;
Xaa₈₀ is Leu or Val or Met;
Xaa₈₂ is Glu or Lys; and
Xaa₈₅ is Glu or Ala;
GLP-2:
His-Ala-Asp-Gly-Ser-Phe-Xaa₁₅₂-Xaa₁₅₃-Xaa₁₅₄-Xaa₁₅₅-Xaa₁₅₆-Xaa₁₅₇-Xaa₁₅₈-Leu-Asp- Xaa₁₆₁-Xaa₁₆₂-Ala-Xaa₁₆₄-Xaa₁₆₅-Xaa₁₆₆-Phe-Xaa₁₆₈-Xaa₁₆₉-Trp-Xaa₁₇₁-Xaa₁₇₂- Xaa₁₇₃-Thr-Xaa₁₇₅-Xaa₁₇₆-Xaa₁₇₇-Xaa₁₇₈;
wherein,
Xaa₁₅₂ is Ser or Thr;
Xaa₁₅₃ is Asp or Ser;
Xaa₁₅₄ is Glu or Asp;
Xaa₁₅₅ is Met or Phe;
Xaa₁₅₆ is Asn or Ser;
Xaa₁₅₇ is Thr or Lys;
Xaa₁₅₈ is Ile or Val or Ala;
Xaa₁₆₁ is Asn or Ile or His or Ser;
Xaa₁₆₂ is Leu or Lys;
Xaa₁₆₄ is Ala or Thr;
Xaa₁₆₅ is Arg or Gln or Lys;
Xaa₁₆₆ is Asp or Glu;
Xaa₁₆₈ is Ile or Leu;
Xaa₁₆₉ is Asn or Asp;
Xaa₁₇₁ is Leu or Ile;
Xaa₁₇₂ is Ile or Leu;
Xaa₁₇₃ is Gln or Asn or His;
Xaa₁₇₅ is Lys or Pro;
Xaa₁₇₆ is Ile or Val;
Xaa₁₇₇ is Thr or Lys; and
Xaa₁₇₈ is Asp or Glu;
Luteinizing Hormone-Releasing Hormone:
Xaa₁-His-Trp-Ser-Tyr-Gly-Leu-Arg-Pro-Gly-OH
wherein,
Xaa₁ is pGlu, 5-oxoPro or Gln.
Melanocyte Stimulating Hormone (MSH):
Ac-Ser-Tyr-Ser-Met-Glu-His-Phe-Arg-Trp-Gly-Lys-Pro-Val-NH₂
Atrial Natriuretic Peptide:
H-Ser-Leu-Arg-Arg-Ser-Ser-Cys-Phe-Gly-Gly-Arg-Xaa₁₃₅-Asp-Arg-Ile-Gly-Ala-Gln-Ser-Xaa₁₄₂-Leu-Gly-Cys-Asn-Ser-Phe-Arg-Tyr-OH
wherein,
Xaa₁₃₅ is Met or Ile; and
Xaa₁₄₂ is Gly or Ser;
Neuromedin B:
H-Gly-Asn-Leu-Trp-Ala-Thr-Gly-His-Phe-Met-NH₂
Human Neuropeptide Y:
H-Tyr-Pro-Ser-Lys-Pro-Asp-Asn-Pro-Gly-Glu-Asp-Ala-Pro-Ala-Glu-asp-Met-Ala- Arg-Tyr-Tyr-Ser-Ala-Leu-Arg-His-Tyr-He-Asn-Leu-Ile-Thr-Arg-Gln-Arg-Tyr-NH₂
Human Orexin A:
pGlu-Pro-Leu-Pro-Asp-Cys-Cys-Arg-Gln-Lys-Thr-Cys-Ser-Cys-Arg-Leu-Tyr-Glu- Leu-Leu-His-Gly-Ala-Gly-Asn-His-Ala-Ala-Gly-Ile-Leu-Thr-Leu-NH₂
Human Peptide YY:
H-Tyr-Pro-Ile-Lys-Pro-Glu-Ala-Pro-Gly-Glu-Asp-Ala-Ser-Pro-Glu-Glu-Leu-Asn- Arg-Tyr-Tyr-Ala-Ser-Leu-Arg-His-Tyr-Leu-Asn-Leu-Val-Thr-Arg-Gln-Arg-Tyr-NH₂
Human Secretin:
H-His-Ser-Asp-Gly-Thr-Phe-Thr-Ser-Glu-Leu-Ser-Arg-Leu-Arg-Glu-Gly-Ala-Arg-Leu-Gln-Arg-Leu-Leu-Gin-Gly-Leu-Val-NH₂
Vasoactive Intestinal peptide (VIP):
H-His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn-NH₂
Antibiotic Peptides such as:
Magainin 1:
Gly-Ile-Gly-Lys-Phe-Leu-His-Ser-Ala-Gly-Lys-Phe-
Gly-Lys-Ala-Phe-Val-Gly-Glu-Ile-Met-Lys-Ser
Magainin 2:
Gly-Ile-Gly-Lys-Phe-Leu-His-Ser-Ala-Lys-Lys-Phe-
Gly-Lys-Ala-Phe-Val-Gly-Glu-Ile-Met-Asn-Ser
Cecropin A:
Lys-Trp-Lys-Val-Phe-Lys-Lys-Ile-Glu-Lys-Val-Gly-
Gln-Ala-Thr-Gln-Ile-Ala-Lys
Cecropin B:
Lys-Trp-Lys-Val-Phe-Lys-Lys-Ile-Glu-Lys-Met-Gly-
Arg-Asn-Ile-Arg-Asn-Gly-Ile-Val-Lys-Ala-Gly-Pro-
Ala-Ile-Ala-Val-Leu-Gly-Glu-Ala-Lys-Ala-Leu.
Substance P (SP):
Arg-Pro-Leu-Pro-Gln-Glu-Phe-Phe-Gly-Leu-Met-amide
Beta Casomorphin-5:
Tyr-Pro-Phe-Pro-Gly
Endomorphin-2:
Tyr-Pro-Phe-Phe-NH2
Procolipase:
100 aa peptide (X1-Pro-X2-Pro-Arg . . . )
Enterostatin:
Val-Pro-Asp-Pro-Arg
Gastrin Inhibitory Peptide:
Tyr-Ala-Glu-Gly-Thr-Phe-Ile-Ser-Asp-Tyr-Ser-Ile-
Ala- Met-Asp-Lys-Ile-His-Gln-Gln-Asp-Phe- Val-
Asn-Trp-Leu- Leu-Ala-Gln-Lys-Gly-Lys-Lys-Asn-Asp-
Trp-Lys-His-Asn-Ile-Thr-Gln
Chromogranin A
Vasostatin I
Vasostatin II:
Leu Pro Val Asn Ser Pro Met Asn Lys Gly Asp Thr
Glu Val Met Lys Cys Ile Val Glu Val Ile Ser Asp
Thr Leu Ser Lys Pro Ser Pro Met Pro Val Ser Gln
Glu Cys Phe Glu Thr Leu Arg Gly Asp Glu Arg Ile
Leu Ser Ile Leu Arg His Gln Asn Leu Leu Lys Glu
Leu Gln Asp Leu Ala Leu Gln Gly Ala Lys Glu Arg
Ala His Gln Gln Lys Lys His Ser Gly Phe Glu Asp
Glu Leu Ser Glu Val Leu Glu Asn Gln Ser Ser Gln
Ala Glu Leu Lys Glu Ala Val Glu Glu Pro Ser Ser
Lys Asp Val Met Glu
Procalcitonin
ProNCT
ProCGRP
Chemokine family:
CXC-group:
IL8 (monocyte-derived):
SerAlaLysGluLeuArgCysGlnCys . . .
GCP-2:
GlyProValSerAlaValLeuThrGluLeuArgCysThrCys . . .
PF4:
GluAlaGluGluAspGlyAspLeuGlnCysLeuCys . . .
IP-10:
ValProLeuSerArgThrValArgCCysThrCys . . .
MIG:
ThrProValValArgLysGlyArgCysSerCys . . .
SDF-1α:
LysProValSerLeuSerTyrArgCysProCys . . .
GRO-α:
AlaProLeuAlaThrGluLeuArgCysGlnCys . . .
1-TAC:
PheProMetPheLysLysGlyArgCysLeuCys . . .
CC-group:
RANTES:
SerProTyrSerSerAspThrThrProCys . . .
LD78:
AlaProLeuAlaAlaAspThrProThrAlaCys . . .
MIP-1α:
AlaProMetGlySerAspProProThrAlaCys . . .
MCP-1:
GlnProAspAlaIleAsnAlaProValThrCys . . .
MCP-2:
GlnProSerAspValSerIleProIleThrCys . . .
MCP-3:
GlnProValGlyIleTAsnSeerThrThrCys . . .
MCP-4:
GlnProAspAlaLeuAspValProSerThrCys . . .
Eotaxin:
GlyProAlaSerValProThrThrCys . . .
MDC:
GlyProTyrGlyAlaAsnMetGluAspSerValCys . . .
and functional analogs and derivatives or fragments thereof.

6. A peptide according to claim 5 wherein the peptide sequence is the sequence of a natural peptide and functional analog or a fragment thereof or a clinically safe and acceptable derivative or analog thereof.

7. A peptide as claimed in claim 1 wherein the peptide sequence is Somatostatin and at least one conformationally rigid moiety is coupled with said somatostatin peptide sequence via an amide bond at different positions as follows:

| Position | conformationally rigid moieties | |
|---|---|---|
| Ala₁ | 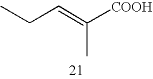 | |
| Asp₅ | 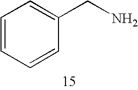 | 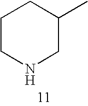 |
| Cys₁₄ | 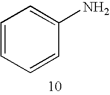 | |
| Ala₁ + Cys₁₄ | 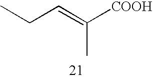 | 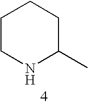 |

8. A peptide as claimed in claim 1 wherein the peptide sequence is PTH 1-34 and at least one conformationally rigid moiety is coupled with said PTH 1-34 peptide sequence via an amide bond at different positions as follows:

| Position | conformationally rigid moieties | |
|---|---|---|
| Ser₁ | 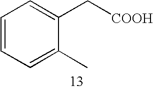 | 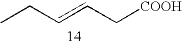 |
| Glu₄ | 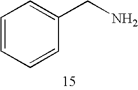 | 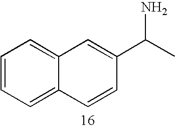 |
| Lys₂₆ | 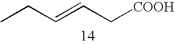 | 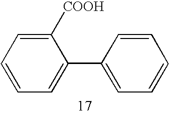 |
| Lys₂₇ | 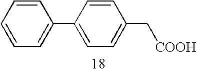 | 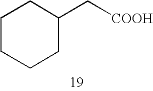 |
| Asp₃₀ | 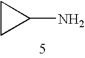 | 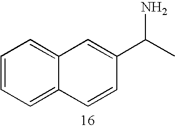 |
| Ser₁ +Lys₂₇ | 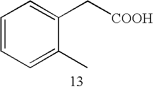 | 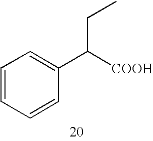 |

9. A peptide as claimed in claim 1 wherein said peptide sequence is GLP-1 and at least one conformationally rigid moiety is coupled with said GLP-1 peptide sequence via an amide bond at different positions as follows:

| Position | conformationally rigid moieties | |
|---|---|---|
| His₁ | 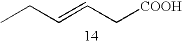 | 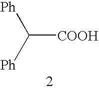 |
| | 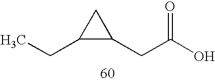 | 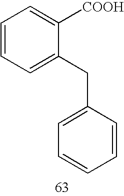 |
| | 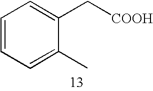 | |
| Glu₃ | 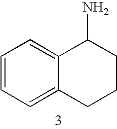 | 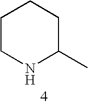 |
| Asp₉ | 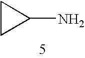 | 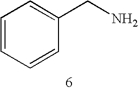 |
| His₁ + Glu₃ | 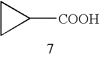 | 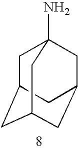 |
| His₁ + Asp₉ | 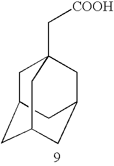 | 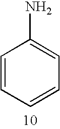 |
| Glu₃ + Asp₉ | 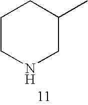 | 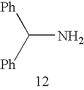 |

10. A peptide as claimed in claim 1 wherein said peptide sequence is GLP-2 and at least one conformationally rigid moiety is coupled with said GLP-2 peptide sequence via an amide or ester bond at different positions of the peptide sequence.

11. A peptide as claimed in claim 1 wherein said peptide sequence is Enterostatin and at least one conformationally rigid moiety is coupled with said Enterostatin peptide sequence via an amide bond at different positions of the peptide sequence.

12. A peptide as claimed in claim 1 wherein said peptide sequence is NPY and at least one conformationally rigid moiety is coupled with said NPY peptide sequence via an amide or ester bond at different positions of the peptide sequence.

13. A peptide as claimed in claim 1 wherein said peptide sequence is NPYY and at least one conformationally rigid moiety is coupled with said NPYY peptide sequence via an amide or ester bond at different positions of the peptide sequence.

14. A peptide as claimed in claim 1 wherein said peptide sequence is Secretin and at least one conformationally rigid moiety is coupled with said Secretin peptide sequence via an amide or ester bond at different positions of the peptide sequence.

15. A peptide as claimed in claim 1 wherein said peptide sequence is Vasoactive Intestinal Peptide and at least one conformationally rigid moiety is coupled with said Vasoactive Intestinal Peptide sequence via an amide or ester bond at different positions of the peptide sequence.

16. A peptide as claimed in claim 1 wherein said peptide sequence is Gastrin Inhibitory Peptide and at least one conformationally rigid moiety is coupled with said Gastrin Inhibitory Peptide sequence via an amide or ester bond at different positions of the peptide sequence.

17. A peptide as claimed in claim 1 wherein said peptide sequence is Vasostatin II and at least one conformationally rigid moiety is coupled with said Vasostatin II peptide sequence via an amide or ester bond at different positions of the peptide sequence.

18. A peptide as claimed in claim 1 wherein said peptide sequence is RANTES and at least one conformationally rigid moiety is coupled with said RANTES peptide sequence via an amide or ester bond at different positions of the peptide sequence.

19. A peptide as claimed in claim 1 wherein said peptide sequence is Eotaxin and at least one conformationally rigid moiety is coupled with said Eotaxin peptide sequence via an amide or ester bond at different positions of the peptide sequence.

20. A peptide as in any one of claims 1 to 18, wherein said conformationally rigid moiety is coupled with said peptide sequence via an amide or ester bond at the N-terminal.

21. A peptide according to any one of claims 8 to 19, wherein the conformationally rigid moiety has the formula 60 referenced in the description.

22. A peptide according to claim 20, wherein the peptide sequence is GLP-1.

23. Use of the peptide according to claim 22 in the treatment of glucose intolerance associated or not with insulin resistance pathologies.

24. Use according to claim 23 in the treatment of type II diabetes.

25. A peptide according to claim 1 wherein said peptide sequence is CGRP and at least one conformationally rigid moiety is coupled with said CGRP peptide sequence via an amide or ester bond at different positions of the peptide sequence.
